(19) United States
(12) Patent Application Publication
(10) Pub. No.: US 2024/0164650 A1
(43) Pub. Date: May 23, 2024

(54) SMARTWATCH-BASED BODY TEMPERATURE MONITORING SYSTEM AND DISEASE PREDICTION METHOD USING THE SAME

(71) Applicants: JangWon LEE, Seoul (KR); Meta Therapeutics Inc., Gwangju (KR)

(72) Inventor: JangWon LEE, Seoul (KR)

(73) Assignees: JangWon LEE, Seoul (KR); Meta Therapeutics Inc., Gwangju (KR)

(21) Appl. No.: 18/377,313

(22) Filed: Oct. 6, 2023

(30) Foreign Application Priority Data
Nov. 17, 2022 (KR) 10-2022-0154916

(51) Int. Cl. A61B 5/01; A61B 5/00; G04G 21/02

(52) U.S. Cl. CPC A61B 5/01; A61B 5/486; A61B 5/681; A61B 5/7221; A61B 5/7275; A61B 5/746; G04G 21/025; G01K 13/20

(57) ABSTRACT

Disclosed is a body temperature monitoring system based on a smart watch, the system including: a baseline measuring unit configured to convert information on body temperature of a user, measured at predetermined intervals, into data; a personal variable input unit configured to store information on age and gender of the user; and a controller configured to compare and analyze a body temperature value converted by the baseline measuring unit and a normal body temperature range derived by the personal variable input unit. The controller is further configured to: determine whether the user's current body temperature falls within the normal body temperature range based on information produced from the baseline measuring unit and the personal variable input unit; and identify a disease type by analyzing a duration, a value, and a timing of deviation of the user's body temperature from the normal body temperature range.

### TECHNICAL FIELD

The present disclosure relates to a body temperature monitoring system based on a smart watch wearable on a user's wrist, and a disease prediction method using the same, and more particularly, a body temperature monitoring system that connects the smartwatch and an application to measure the user's basic body temperature, send a notification signal in response to detection of abnormal body temperature, and predict a disease through analysis of an abnormal temperature range, and a disease prediction method using the same.

### BACKGROUND

Recently, respiratory infectious diseases such as COVID-19 have been increasingly prevalent worldwide. In particular, fever may be considered as a prominent symptom associated with these infectious diseases, so measuring body temperature has become essential for individuals to determine their infection status. Therefore, not only in hospitals but also in homes and workplaces, the presence of thermometers has become commonplace, and there is a growing trend for individuals to pay more attention to the health management.

Furthermore, in households where families are raising infants or women are managing their pregnancy cycles by measuring basal body temperature, the presence of thermometers is even more crucial. Accordingly, there is a growing demand for products that enable accurate temperature measurement, precise assessment of bodily anomalies through temperature readings, and individual health management through analysis of recorded temperature data.

In this regard, Korean Patent Application Publication No. 2022-0063352 (Related Document 1) discloses a temperature measurement device that can be easily detachable from a user's body or mask to conveniently measure temperature of the user in real time, and a management system including the same. According to this related document, users can continuously measure their body temperature and other vital signs using the temperature measurement device, and integrate the device into everyday items, thereby reducing the risk of product loss.

In another example, Korean Patent Application Publication No. 2020-0021711 (Related Document 2) discloses a patch-type thermometer-based body temperature management system that can be easily attached to a user's body to conveniently measure the user's temperature and automatically transmit measurement information to a temperature management server, and a temperature management method thereof. According to this related document, body temperature-related information may be transmitted to a terminal carried by the user or guardian while the patch-type thermometer is attached to the user's skin.

However, the temperature measurement devices and systems disclosed in the aforementioned related documents have the drawback of being unable to accurately measure body temperature because they do not take into account factors such as individual characteristics, daily rhythms, hormonal changes, and other variables that affect temperature measurements. In particular, the temperature measurement devices disclosed in the related documents simply measure the user's temperature and record the measurement, thus having a fundamental limitation in that they cannot account for various environmental factors and the user's personal variables.

Furthermore, the existing temperature measurement systems only address basic temperature measurement methods and emergency response procedures in the event of abnormal temperatures, without disclosing a technology for predicting diseases based on these measurements. Therefore, in light of the prevailing societal concern regarding respiratory infections, the existing technologies have primarily catered to the demand for temperature measurement alone, falling short of meeting the market demand for disease prediction based on temperature information.

### RELATED DOCUMENTS

### Patent Documents

Related Document 1: Korean Patent Application Publication No. 2022-0063352 (Published on May 17, 2022)
Related Document 2: Korean Patent Application Publication No. 2020-0021711 (Published on 2020 Mar. 2)

### SUMMARY

The present disclosure provides a body temperature monitoring system capable of more accurately measuring body temperature of a user and predicting a disease based on measured body temperature information, and a disease prediction method using the same. An object of the present disclosure is to provide a smartwatch-based body temperature monitoring system capable of performing accurate temperature measurements by adjusting for variables occurring during the user's temperature measurement, and using measured temperature information to identify the type of disease the user may have contracted, and a disease prediction method using the system.

In one aspect of the present disclosure, there is provided a body temperature monitoring system based on a smart watch, the system comprising: a baseline measuring unit configured to convert information on body temperature of a user, measured at predetermined intervals, into data; a personal variable input unit configured to store information on age and gender of the user; and a controller configured to compare and analyze a body temperature value converted by the baseline measuring unit and a normal body temperature range derived by the personal variable input unit. The controller is further configured to: determine whether the user's current body temperature falls within the normal body temperature range based on information produced from the baseline measuring unit and the personal variable input unit; and identify a disease type by analyzing a duration, a value, and a timing of deviation of the user's body temperature from the normal body temperature range.

In another aspect of the present disclosure, there is provided a disease prediction method, the method comprising: storing reference data on body temperature of a user at predetermined intervals in a smart watch; measuring information on the body temperature of the user in real time using the smart watch; comparing the reference data stored in the smart watch with information on the body temperature of the user measured in real time; in response to the information on the user's body temperature falling outside a range of the reference data, analyzing information on an abnormal signal by a disease type analysis unit; and determining, by a controller, a disease type based on information on a duration of the abnormal signal, an abnormal value, and a time of occurrence of the abnormal signal, which are derived by the disease type analysis unit.

According to the proposed present disclosure, it is possible to derive more accurate body temperature data compared to conventional simple body temperature measuring devices because information on basic body temperature of a user is extracted in consideration of the user's circadian rhythm, hourly body temperature changes, hormones, and the like and the user's final body temperature value is corrected in consideration of various variables in the basal body temperature.

In addition, according to the present disclosure, it is possible to enable accurate body temperature measurement regardless of environmental factors because body temperature variables such as information on a body location for temperature measurement, information on physical activity, and stress-related information are used as factors for correction of a body temperature value.

In addition, according to the present disclosure, it is possible to meet a market demand for disease prediction based on measured body temperature information because information on a disease infected with the user is derivable based on a final body temperature value calculated by adjusting various variables.

In addition, according to the present disclosure, it is possible to enable efficient health management for the user because the user is able to receive an alert regarding a disease and countermeasures for the disease through an alert signal.

### BRIEF DESCRIPTION OF THE DRAWINGS

FIG. 1 is a block diagram illustrating how a user's body temperature measurement result is accurately derived in consideration of each individual's variables according to an embodiment of the present disclosure.

FIG. 2 is a block diagram illustrating how a user's body temperature measurement result is derived more accurately in consideration of environmental variables that occur when measuring the user's body temperature.

FIG. 3 is a flowchart overall showing the process of measuring the user's body temperature through a smart watch and the process of predicting a disease based on measured body temperature information.

FIG. 4 is a flowchart for providing a detailed explanation of a process of generating reference data on a user's body temperature in the smart watch.

FIG. 5 is a diagram showing an example of a smart phone screen showing how information on a user's body temperature, information on a disease predicted based on measured body temperature, and a method of coping with the disease are displayed on a user's smart phone.

FIG. 6 is an example diagram illustrating the temporal progression of a specific disease using a smartwatch and an application according to the present disclosure.

### DETAILED DESCRIPTION

Since various modifications may be made to the present disclosure and the present disclosure may have various forms, specific embodiments will be illustrated and explained in detail. However, it should be understood that it is not intended to limit the present disclosure to specific disclosed forms, and all the modifications, equivalent or replacements within the scope of the idea and technology of the present disclosure are included. The same reference numerals designate the same components in the drawings.

Unless otherwise defined, all terms including technical and scientific terms used herein have the same meanings as those commonly understood by one of ordinary skill in the art to which the present disclosure belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Hereinafter, embodiments of the present disclosure will be described in more detail with reference to the drawings.

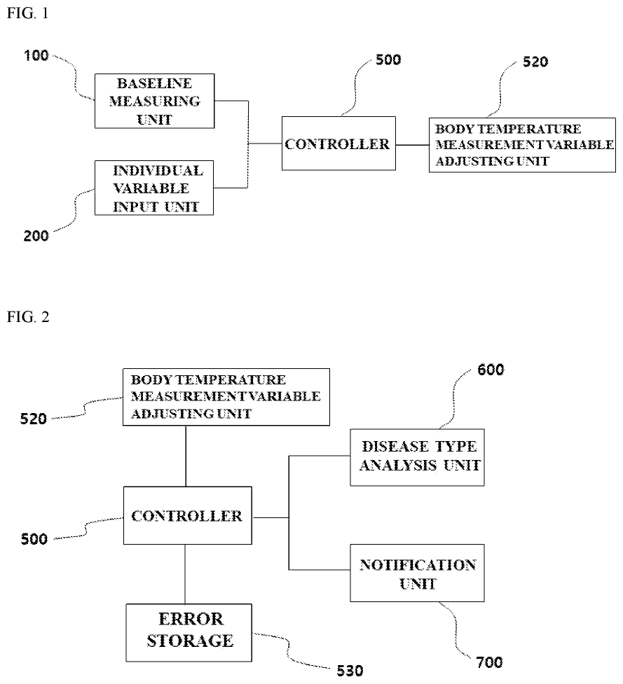

FIG. 1 is a block diagram illustrating how a user's body temperature measurement result is accurately derived in consideration of each individual's variables according to an embodiment of the present disclosure. In addition, FIG. 2 is a block diagram illustrating how a user's body temperature measurement result is derived more accurately in consideration of environmental variables that occur when measuring the user's body temperature.

Referring to FIGS. 1 and 2, in a method for measuring a user's body temperature according to an embodiment of the present disclosure, the user's body temperature may be measured using a smart watch that the user can wear on a part of the body, such as the wrist. Information such as body temperature measured using the smart watch may be transmitted to the user's electronic device over a communication network. The kind of the electronic device is not limited as long as the device is capable of communication, such as a smart phone, computer, or tablet PC.

In the smart watch, the user's body temperature may be continuously measured, and measured body temperature data may be analyzed at predetermined intervals, for example, on a daily or monthly basis. Specifically, the body temperature data may be measured as through following a consistent pattern based on each individual's characteristics. For example, while the activity hour and sleep time vary for each individual, it is common for the activity hour and sleep time to remain consistent when considering each individual separately. Such body temperature data for each individual may be stored in a baseline measuring unit 100. In other words, the baseline measuring unit 100 is configured to store information about baseline data for each individual's body temperature, taking into account their daily and monthly rhythm cycles. Accordingly, when a user wears the smart watch for at least two months, for example, accurate information on reference data for each individual may be stored in the baseline measuring unit 100.

In addition, each user may check a normal body temperature range for each individual using a personal variable input unit 200. For example, the personal variable input unit 200 may store fixed information values about the user's age and gender. In general, infants have a relatively higher body temperature than adults. Therefore, the range for determining a normal body temperature varies by age. From a medical perspective, body temperature of up to 38 degrees Celsius is considered within the normal range for infants up to 2 years old, while for individuals aged 11 and above, typically considered as adults, a normal body temperature range of up to approximately 37.5 degrees Celsius is considered within the normal range. In addition, women may experience relatively more significant fluctuations in body temperature compared to men, and during their menstrual cycle, hormonal changes may lead to temperature variations distinct from those in men. Based on this information, the normal body temperature range for each individual may be determined. As such, the personal variable input unit 200 may be characterized in that the user directly inputs his or her information into an application, system, or the like.

Information derived from the baseline measuring unit 100 and the personal variable input unit 200 is transmitted to a controller 500. Specifically, the controller 500 receives reference data on individual body temperature extracted from the baseline measuring unit 100. Next, the controller 500 receives information on the user's age and gender received from the personal variable input unit 200 and sets information on a normal body temperature range. In particular, the controller 500 may correct the individual reference data by adding data input from the personal variable input unit 200.

In addition, the smart watch wearable on the user's wrist may also measure the user's blood pressure, heart rate, breathing rate, and the like. However, in order to measure the user's body temperature more accurately, it is necessary to consider factors in the user's surrounding environment. Taking these factors into account, the controller 500 may communicate with a body temperature measurement variable adjusting unit 520 that stores information capable of serving as variables in the user's body temperature measurement. For example, the variables considered by the body temperature measurement variable adjusting unit 520 may include information on a body location for temperature measurement, information on physical activity, stress-related information, temperature measurement data taking into account an oxygen dissociation curve, information on a decreasing trend in human body temperature, and information on environment temperature.

The information on a body location of temperature measurement is data for correcting body temperature in consideration the fact that a person's temperature varies depending on a body part. Specifically, when it comes to the average temperature range for different parts of the human body, the forehead temperature is approximately 36.6° C. to 37.9° C., the ear temperature is approximately 35.8° C. to 37.5° C., the oral temperature is approximately 35.5° C. to 37.5° C., and the armpit temperature is approximately 35.7° C. to 37.3° C. Since body temperature varies slightly in each part of the human body, information on a body location of temperature measurement may be an important variable to consider when measuring body temperature. In particular, according to the present disclosure, since the skin of the wrist, that is, the extremity of the body is measured at a body part where the smart watch is worn, correction for a body temperature value at the body part is necessary.

As for the information on physical activity, when a user performs a sudden physical activity, such as exercise, there is an increase in heat production as carbohydrates and fats are broken down for energy generation. Accordingly, if the user measures body temperature after performing the sudden physical activity, a high temperature outside the normal range may be detected during the measurement of the body temperature, even though the user's health condition is not compromised. Therefore, the body temperature measurement variable adjustment unit 520 needs to transmit the information on physical activity to the controller 500, enabling a more accurate body temperature measurement.

The stress-related information is generated when the sympathetic nerves are stimulated when the user experience emotional or physical stress. If the sympathetic nerves are stimulated, it leads to an increase in the secretion of epinephrine and norepinephrine within the body, and as a result of increased adrenal activity, heat production also rises. Therefore, the body temperature measurement variable adjusting unit 520 needs to transmit the stress-related information to the controller 500, enabling a more accurate body temperature measurement.

Body temperature measurement information taking into account the oxygen dissociation curve is based on an increase in a user's body temperature, accompanied by an increase in respiratory rate and heart rate, may be considered a significant temperature change. Generally, oxygen in the blood is mostly transported by binding with the hemoglobin, a protein that makes up red blood cells. The oxygen dissociation curve is a graph showing the correlation between the partial pressure of oxygen in the plasma and the oxygen saturation of hemoglobin. The oxygen saturation of hemoglobin is a value representing the amount of oxygen bound to hemoglobin.

If the body temperature rises, the oxygen saturation of hemoglobin decreases, causing the oxygen dissociation curve to shift to the right, which indicates a decrease in the oxygen saturation of hemoglobin at the same oxygen partial pressure. Furthermore, in cases of heightened metabolism, such as during infections or inflammation, oxygen may more easily detach from hemoglobin. In this manner, when the oxygen saturation is reduced, a person's respiratory rate and heart rate increase to meet the oxygen demands.

A body temperature measurement correction variable of the present disclosure may ultimately identify only the significant temperature changes in the user, which are accompanied by an increase in heart rate and respiratory rate. In other words, it may be challenging to accurately discern phenomena, such as precise body temperature measurement results or disease infection information, solely based on the simple occurrence of an increase in body temperature. Therefore, in the present disclosure, only when both the heart rate and the respiratory rate increase simultaneously, the correction variable indicating that a user's body temperature has changed may be utilized, and procedures such as disease prediction may be performed accordingly.

The information on a decreasing trend in human body temperature is based on the observation that the body temperature of individuals measured in specific regions at particular times gradually exhibits a decreasing trend. According to recent research results in major countries such as the United States and the United Kingdom, it has been observed that the body temperatures of individuals in the same regions are showing a decreasing trend over time. For example, in a study conducted in 2017, involving 35,000 adults residing in a specific region of the United Kingdom, the average body temperature of the adults in the specific region was measured to be 36.6° C. (97.9° F.). However, in 2019, the average body temperature of adults residing in the specific region was measured at 36.4° C. (97.5° F.).

Such a decreasing trend of body temperature is attributed to several hypotheses, including improved hygiene conditions, access to clean water due to technological advancements, vaccination, and advancements in the field of medicine. Furthermore, the decrease in people's body temperature may be attributed to the facts that even when individuals contract infectious diseases, preparations for combating infections may be more easily undertaken and that overall infection durations may be reduced due to easier access to antibiotics and other treatments. For these complex reasons, people's body temperature tends to gradually decrease.

In the present disclosure, with this trend taken into account, a process is conducted to statistically analyze the overall trend of body temperature decrease in individuals measured in the same region at the same time so as to adjust an associated variable. In other words, based on users wearing smartwatches, the body temperature decreasing trend of individuals measured in a specific region at a specific time is statistically analyzed and adjusted with a variable related to a normal body temperature range. Accordingly, in the present disclosure, the tendency of a person's body temperature to decrease over time may be taken into account, so that the tendency can be utilized as a variable for adjusting reference data, for example, to establish a more accurate standard body temperature range.

For the information on environment temperature, for example, when a user enters indoors directly from a cold place and measures his or her body temperature, naturally, a low body temperature, rather than the normal body temperature, may be recorded. Considering the above, the controller 500 determines the user's location information through positioning methods using GPS, satellite signals, Wi-Fi, Bluetooth, and the like mounted on a smart watch or the user's smart phone. Next, based on the determined location information, the controller 500 may adjust an indoor temperature variable by statistically analyzing body temperature variances of other individuals measured at the same location. In this manner, the body temperature measurement variable adjusting unit 520 needs to transmit information on external environment temperature to the controller 500, enabling a more accurate body temperature measurement.

Based on a signal received from the body temperature measurement variable adjusting unit 530, the controller 500 finally determines whether the user's body temperature falls within the normal range. If the user's body temperature falls outside the normal range, the controller 500 may communicate with a disease type analysis unit 600, which stores information on disease types based on the duration, value, and timing of deviation of the user's body temperature from the normal range.

The disease type analysis unit 600 is configured to serve as a database where various information on disease types, symptoms, timings, and the like are stored in advance. For example, in the case of diseases like the common cold or pneumonia, fever manifests in a pattern of repeatedly alternating between the normal range and high temperatures, while bacterial infections such as typhoid fever or meningitis exhibit a pattern of gradually rising temperatures that persist all day. In addition, when infected with malaria, fever occurs in a cycle of rising and falling every one to two days, and tuberculosis exhibits a specific temperature fluctuation pattern where fever only occurs during the night. As such, various diseases have distinct temperature change patterns.

The controller 500 compares and analyzes the final data on the user's body temperature with the data on diseases stored in the disease type analysis unit 600. Accordingly, the controller 500 may determine a disease type by analyzing the duration, value, and timing of deviation of the user's body temperature from the normal range.

Hereinafter, a method for measuring a user's body temperature and an algorithm for predicting a disease will be described in detail step by step.

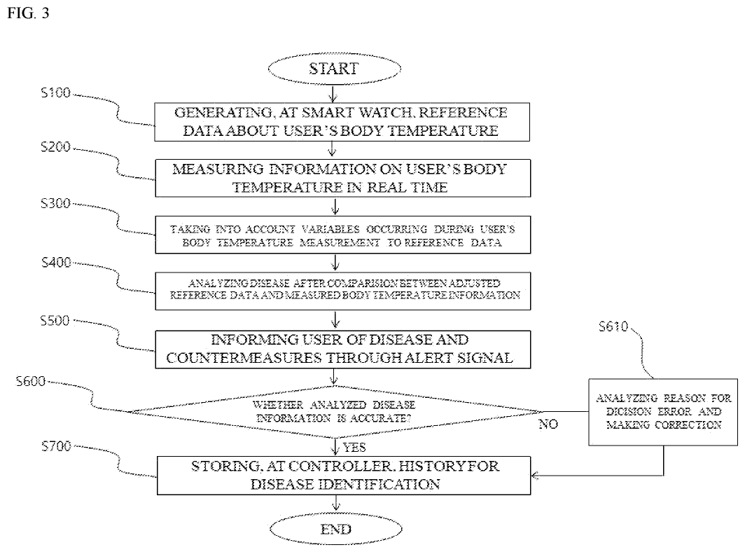

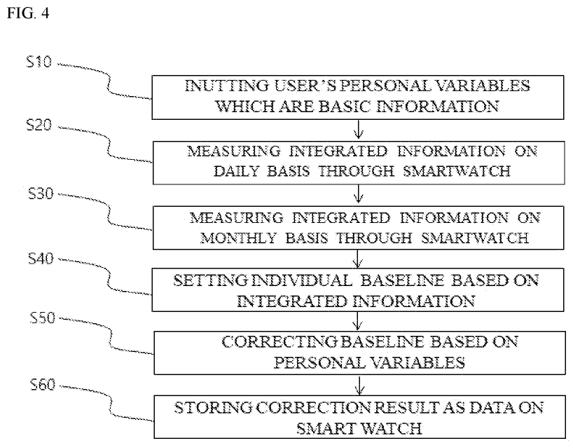

FIG. 3 is a flowchart overall showing the process of measuring the user's body temperature through a smart watch and the process of predicting a disease based on measured body temperature information. In addition, FIG. 4 is a flowchart for providing a detailed explanation of a process of generating reference data on the user's body temperature in the smart watch.

With reference to the drawings, first, the user wears the smart watch to measure his or her body temperature. When the user wears the smart watch for more than a predetermined period, for example, more than two months, the smart watch generates reference data about the user's body temperature (S100).

A process of generating reference data about the user's body temperature is shown in detail in FIG. 4. First, the user enters information on his or her age and gender into a system (S10). As a result, information on a normal range of the user's body temperature is set in the system. Next, when the user wears the smart watch for more than the predetermined period of time, the user's individual daily pattern (S20) and monthly pattern (S30) of temperature changes may be extracted. The daily pattern may include a pattern based on the user's sleep cycle, a pattern based on the user's basal metabolic rate, and a pattern based on the user's hourly temperature fluctuations. In addition, a representative example of the monthly pattern may be a pattern based on hormonal changes due to phenomena like ovulation or menstruation.

After extracting the user's individual daily and monthly pattern of body temperature changes, a baseline for the user is set based on the integrated information (S40). The baseline may refer to reference data about each individual's body temperature, derived from the analysis of patterns of changes in body temperature of each individual. Next, the set individual baseline may be corrected by taking into account information on the user's age and gender provided at the outset (S50). A result corrected according to this process may be stored as the user's reference data in the smart watch (S60).

Referring back to FIG. 3, when operation S100 is completed, the smart watch proceeds with a process of measuring the user's current body temperature in real time (S200). However, if there are variables that can affect body temperature, such as after the user has engaged in vigorous exercise, operation S200 may not proceed for a predetermined period.

When a signal indicating that the user's condition is stable is sent, the controller 500 and the body temperature measurement variable adjusting unit 520 communicate with each other (S300) in order to exclude any external environmental factor related to the user's body temperature measurement. By taking into account information on a body location for temperature measurement, information on physical activity, stress-related information, temperature measurement data taking into account an oxygen dissociation curve, information on a decreasing trend in human body temperature, and information on environment temperature which are used as judgment variables in the body temperature measurement variable adjusting unit 520, variables occurring during the user's body temperature measurement are reflected in the reference data.

Next, the controller 500 compares the user's body temperature measured in a stable state with the reference data (S400). In other words, a process is conducted to determine whether the user's body temperature measured by the smart watch falls within a normal range.

If the user's body temperature is determined to falls outside the normal range according to a result of comparison between the adjusted reference data and the measured body temperature information, the controller 500 identifies a disease type by analyzing the duration, value, and timing of deviation of the user's body temperature from the normal range. In this case, the controller 500 may communicate with the disease type analysis unit 600 to match a disease type based on the user's body temperature trend.

When the analysis of the disease type is completed, the controller 500 sends a notification signal about a disease to the user as an alert signal and provides countermeasures for the disease (S500). Accordingly, the user may verify what disease he or she has been exposed to and efficiently perform emergency measures for the verified disease.

Next, the controller 500 determines whether the analyzed disease information is accurate (S600). In the future, if the user visits a hospital or medical facility for treatment, and if the analysis of a disease type by the controller 500 yields a different result, the user may suspect an issue with the system. Then, the controller 500 analyzes a reason for such a decision error and carries out a process to correct associated data (S610). In addition, a process of analyzing the reason for the decision error and correcting the associated data may be recorded in an error storage 530. The controller 500 communicates with the error storage 530, so that the system can properly learn to prevent repeated errors.

When it is determined that the disease information analyzed in operation S600 are accurate, the controller 500 stores the entire history from the body temperature measurement process to the disease prediction process (S700). As various information accumulates in the controller 500 over time, the controller 500 may perform more accurate and efficient disease predictions for similar body temperature patterns.

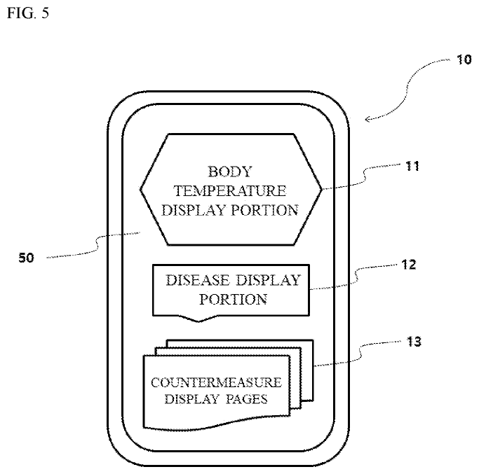

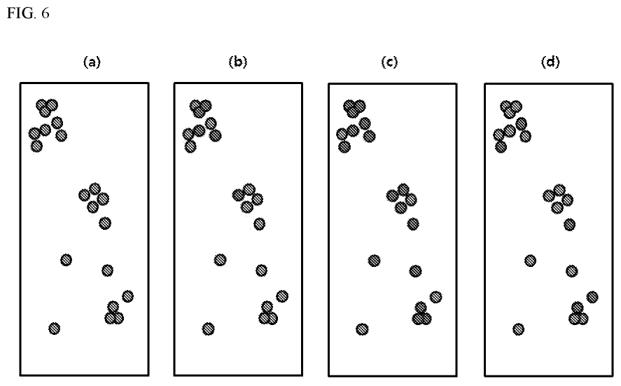

FIGS. 5 and 6 show examples of how the present disclosure is applied to a smartphone and a real-life situation. First, FIG. 5 is a diagram showing an example of a smart phone screen showing how information on a user's body temperature, information on a disease predicted based on measured body temperature, and a method of coping with the disease are displayed on a user's smart phone.

As shown in FIG. 5, the user's smart watch is linked to the user's smart phone 10 so that data can be transmitted and received. That is, information on reference data about the user's body temperature is stored in the smart phone 10 and the smart watch. Therefore, when a user measures his or her current body temperature using a smart watch, it may be determined whether the user's current body temperature falls within a normal range of the reference data by taking into account various variables. This information on body temperature is displayed through a display 50 of the smart phone. For example, a body temperature information display portion 11 is formed at an upper part of the display 50. The body temperature information display portion 11 may display information on a precise body temperature measurement time, the user's body temperature, external temperature, and the like.

If it is determined that the user's body temperature falls outside the normal range, the disease type is identified by analyzing the duration, value, and timing of deviation of the user's body temperature from the normal range. In addition, the controller 500 communicates with a notification unit 700, which transmits an alert signal to the user, and provides the user with guidance on the disease type and countermeasures for the disease. For example, when a disease type analysis result indicates bronchitis, the notification unit 700 may perform a notification for bronchitis and provide information on countermeasures and emergency measures for bronchitis.

As such, information on a disease and information on countermeasures for the disease may be displayed on the display 50 of the smart phone. Specifically, a disease display portion 12 and display pages 13 about the countermeasures for the corresponding disease are sequentially formed below the body temperature information display portion 11. In addition, the user may check emergency measures for a suspected diseases in more detail by flipping through the countermeasures display pages 13.

A body temperature measurement application according to a related art require a user to manually input his or her body temperature, and the target age for temperature measurement is limited to children. However, the body temperature measurement application according to the present disclosure may be linked to a smart watch to automatically display a body temperature measurement value, a disease indication, and countermeasures. In addition, since the application of the present disclosure is linked to a smart watch, there is the advantage of applicability to all ages, from children to adults, for temperature measurement.

FIG. 6 is an example diagram illustrating the temporal progression of a specific disease using a smartwatch and an application according to the present disclosure. In this case, a person to be investigated for infection with a specific disease is limited to a user wearing a smart watch according to the present disclosure.

For example, FIG. 6 illustrates information on the distribution of specific disease infections within a designated region, displayed in a rectangular shape. Green circles within the designated region may represent the distribution of individuals not infected with the specific disease, and red circles may represent the distribution of individuals infected with the specific disease. In the drawing, at time (a), it can be said that the specific disease has not yet spread within the designated region. However, from time (b), the specific disease begins to manifest, primarily in the upper-left area of the designated region, and by time (c), the specific disease becomes prevalent throughout the designated region. In other words, at a time like (c), it can be said to be a pandemic state of the specific disease. However, as solutions such as treatment methods or vaccines for the specific disease are introduced, it can be seen that the specific disease is gradually disappearing at time (d).

As such, according to the present disclosure, since information on a disease that a user may have contracted can be derived based on a final body temperature value calculated by adjusting various variables, it is possible to meet a market demand for disease prediction based on measured body temperature information.

Although the present disclosure has been particularly shown and described with reference to exemplary embodiments thereof, it will be understood by those of ordinary skill in the art that various modifications and changes may be made therein without departing from the spirit and scope of the present disclosure as defined by the following claims.

### DETAILED DESCRIPTION OF MAIN ELEMENTS

100: base line measuring unit
200: personal variable input unit
500: controller
520: body temperature measurement variable adjusting unit
530: error storage
600: disease type analysis unit
700: notification unit

What is claimed is:

1. A body temperature monitoring system based on a smart watch, the system comprising:
a baseline measuring unit configured to convert information on body temperature of a user, measured at predetermined intervals, into data;
a personal variable input unit configured to store information on age and gender of the user; and
a controller configured to compare and analyze a body temperature value converted by the baseline measuring unit and a normal body temperature range derived by the personal variable input unit,
wherein the controller is further configured to:
determine whether the user's current body temperature falls within the normal body temperature range based on information produced from the baseline measuring unit and the personal variable input unit; and
identify a disease type by analyzing a duration, a value, and a timing of deviation of the user's body temperature from the normal body temperature range.

2. The body temperature monitoring system of claim 1, wherein in the determining of whether the user's current body temperature falls within the normal body temperature range, the controller communicates with a body temperature measurement variable adjusting unit, which takes into account, judgement variables, information on a body location for temperature measurement, information on physical activity, stress-related information, temperature measurement data taking into account an oxygen dissociation curve, information on a decreasing trend in human body temperature, and information on environment temperature.

3. The body temperature monitoring system of claim 1, wherein the controller communicates with a disease type analysis unit where information on a disease type based on the duration, value, and time of deviation of the user's body temperature from the normal body temperature range are stored in advance.

4. The body temperature monitoring system of claim 3, wherein when the disease type analysis unit determines a disease type of the user, the controller communicates with a notification unit, which sends an alert signal to the user, to guide the user with the disease type and countermeasures for the disease.

5. The body temperature monitoring system of claim 1, wherein when the body temperature measurement variable adjusting unit sends a signal indicating that adjustment of the user's body temperature measurement variables has been completed, the controller compares and analyzes the body temperature value converted by the baseline measuring unit and the normal body temperature range derived by the personal variable input unit.

6. A disease prediction method, the method comprising:
storing reference data on body temperature of a user at predetermined intervals in a smart watch;
measuring information on the body temperature of the user in real time using the smart watch;
adjusting the reference data by taking into account information on the user's body temperature measurement variables that comprise information on a body location for temperature measurement, information on physical activity, stress-related information, temperature measurement data taking into account an oxygen dissociation curve, information on a decreasing trend in human body temperature, and information on environment temperature;
in response to the reference data being adjusted in consideration of the body temperature measurement variables, comparing the adjusted reference data with information on the body temperature of the user measured in real time;
in response to the information on the user's body temperature falling outside a range of the reference data, analyzing information on an abnormal signal by a disease type analysis unit; and
determining, by a controller, a disease type based on information on a duration of the abnormal signal, an abnormal value, and a time of occurrence of the abnormal signal, which are derived by the disease type analysis unit.

7. The disease prediction method of claim 6, wherein when the determining of the disease type is completed, the controller transmits a notification about the disease type and guidance on countermeasures to the user.

8. The disease prediction method of claim 7, further comprising:
after the transmitting of the notification about the type of the disease, determining whether information on a disease informed to the user is accurate,
wherein when the information on the disease is inaccurate, the controller further analyzes and corrects a reason for a decision error.
